(19) United States
(12) Patent Application Publication
(10) Pub. No.: US 2003/0044870 A1
(43) Pub. Date: Mar. 6, 2003

(54) Generic capture elisa using recombinant fusion proteins for detecting antibodies in biological samples

(76) Inventors: Peter Sehr, Heidelberg (DE); Michael Pawlita, Eschelbronn (DE); Christof Gaenzler, Nussloch (DE)

Correspondence Address: HELLER EHRMAN WHITE & MCAULIFFE LLP, 1666 K STREET,NW, SUITE 300, WASHINGTON, DC, 20006, US

(21) Appl. No.: 10/193,685

(22) Filed: Jul. 12, 2002

Related U.S. Application Data
(60) Provisional application No. 60/305,259, filed on Jul. 13, 2001.

(51) Int. Cl. G01N 33/53; G01N 33/537; G01N 33/543; B05D 3/00

(52) U.S. Cl. 435/7.93; 427/2.11

(57) ABSTRACT

The present invention relates to a generic capture ELISA for the detection and measurement of antibodies in biological fluids such as serum. This newly developed enzyme-linked immunosorbent assay (ELISA) system uses a first binding partner of a binding pair, preferably glutathione, crosslinked to casein as capture protein to bind recombinant protein antigens fused to a second binding partner of said binding pair, preferably N-terminal glutathione S-transferase (GST). The method not only allows the specific and efficient detection of antibodies in biological samples but, in addition, simple and efficient immobilization and one-step purificaton of overexpressed recombinant antigens even from crude lysates on ELISA plates coated with the first binding partner/casein. Several antigens can be tested in parallel under the same conditions without the need to biochemically purify or renature the proteins.

### FIELD OF THE INVENTION

The present invention relates to a generic capture ELISA for detecting and/or quantifying antibodies in biological samples.

### BACKGROUND OF THE INVENTION

The present invention provides a specific and sensitive method of detecting antibodies in biological samples. This capture ELISA was developed in particular, for the detection of antibodies to recombinant GST-fused polypeptides in serum. This method, however, is generally applicable to other biological samples such as plasma, saliva, urine, milk, semen, tears, lymph, spinal fluids, ascites, peritoneal and other effusions and vaginal secretions etc. and also to laboratory fluids such as tissue culture supernatans or cell lysates.

Enzyme-linked immunosorbent assays (ELISA) for the detection of antibodies to protein antigens often use direct binding of short synthetic peptides to the plastic surface of a microtitre plate. The peptides are, in general, very pure due to their synthetic nature and efficient purification methods using high-performance liquid chromatography. A drawback of short peptides is that they usually represent linear, but not conformational or discontinuous epitopes. To present conformational epitopes, either long peptides or the complete native protein are used. Direct binding of the protein antigens to the hydrophobic polystyrene support of the plate can result in partial or total denaturation of the bound protein and loss of conformational epitopes. Coating the plate with an antibody, which mediates the immobilization (capture ELISA) of the antigens, can avoid this effect. However, frequently, overexpressed recombinant proteins are insoluble and require purification under denaturing conditions and renaturation, when antibodies to conformational epitopes are to be analyzed.

As an alternative approach, in the present invention recombinant protein antigens were overexpressed as fusion proteins with glutathione S-transferase (GST) in E. coli. This approach has a variety of advantages. The directed crosslinking in solution of a first binding partner of a binding pair, e.g. glutathione, to casein allows the production of a large batch of homogeneous capture protein sufficient in quantity to coat hundreds or thousands of ELISA plates. With aliquots of the same batch of capture protein, high reproducibility of the coat can be achieved at least after up to 1 year of storage at −20° C. Coupling of glutathione to hemoglobin precoated on plates as described previously is clearly more demanding and is associated with larger variations and less reproducibility: The plates must be prepared anew each time and tested for their properties. It is problematic to control the chemical reaction of the water-soluble crosslinker SSMPB and the multistep pipetting for each well could increase well-to-well variability.

The generation of a capture protein for, e.g. GST, by crosslinking, e.g. glutathione, to casein has several advantages: Low background reaction with specific antibodies in biological fluids of many species, especially humans; absence of or at least low binding activities with other proteins, high solubility and native conformation of the purified protein antigen in physiological buffer systems and high stability during longterm storage. Moreover, glutathione-coated plates from commercial distributors are restricted in terms of the protein carrier, which can be problematic in some applications and associated with nonspecific binding and high background reactivity.

The capture ELISA of the present invention is independent of the biological source of the sera, whereas an ELISA using a capture antibody cannot be used with sera from the species providing the capture antibody. Thus, the same capture ELISA can serve as a screening tool in vaccination trials in any animal model, as well as in serological studies with human sera.

When using glutathione as a binding partner; only native GST can bind to glutathione, so that glutathione-mediated capture selects for soluble proteins with a native conformation at least in the GST portion. Due to the high specific binding of glutathione to GST, one can conveniently immobilize and purify GST fusion proteins from crude lysates in one step on glutathione casein-coated plates as demonstrated in the Examples, below, with E6 and E7 proteins of HPV 16 and 18.

The format of the ELISA of the present invention allows parallel testing of a series of antigens under homogeneous, standardized conditions with only one background control (e.g. GST-TAG) for all antigens. Potential antibody reactivities with contaminating bacterial proteins are successfully blocked by preincubating the sera with lysate from wild-type bacteria of the same strain. This blocking of sera against bacterial contamination appears to be more important for larger, potentially E. coli protein-binding (“sticky”) proteins, such as HPV E2 proteins than for the small E6 and E7 proteins. Further, background reactions with, e.g. the GST or the TAG portions of the fusion proteins can be suppressed by preincubating the sera with lysate from bacteria overexpressing GST-TAG fusion protein.

With the capture ELISA of the present invention, it is possible to bind antigen at a higher density to the plate as in the antibody capture ELISA and so to increase the sensitivity of the method. The relatively small glutathione casein (MW 30 kDa) possesses 12 potential binding sites for GST fusion proteins/casein molecule if every lysine is crosslinked to glutathione, whereas antibodies (IgG, MW=about 150 kDa) exhibit only two binding sites per molecule for capturing the antigen. It was observed that more GST-X-TAG protein from E. coli lysates could be bound to glutathione casein-coated plates than to mouse anti-TAG coated plates. A densitometric quantification of a silver-stained SDS gel of probes eluted from these plates (as in FIG. 5) showed that 1.3-fold more GST16E6TAG, 2.8-fold more GST16E7TAG, 4.0-fold more GSTI8E6TAG and 1.8-fold more GSTI8E7TAG is bound to the glutathione casein-coated plate than to the anti-TAG coated plate. Higher antigen density on the plates could be one reason for the higher sensitivity of the capture ELISA of the present invention compared to the TAG capture ELISA. Alternatively, a different presentation of C- and N-terminal epitopes could contribute to the discordant reactivity of some sera. In both assay formats, the antigens carry the small TAG peptide at their C-terminus. However, in the TAG capture ELISA, it is bound by the bulky anti-TAG antibody, which might interfere with other antibodies binding to C-terminal epitopes of the HPV protein sequences. In contrast, in the capture ELISA of the present invention, such epitopes might be freely accessible. Conversely, in the capture ELISA, the N-terminus of the antigen is fused to the binding partner, e.g. GST, which might influence the accessibility of N-terminal epitopes freely accessible in the TAG capture ELISA.

Overall, a good correlation of antibody reactivities could be demonstrated in both ELISA procedures. Taken together, the capture ELISA of the present invention with antigens from crude E. coli lysates is not only much easier to perform than the sophisticated antibody capture ELISA with biochemically purified and renatured yeast proteins, it is also more sensitive. In general, the ELISA plates prepared according to the present invention offer a cheap, convenient means for capturing fusion proteins, e.g. GST fusion proteins, from lysates of E. coli (or other expression systems), e.g., as antigens in serological studies, as well as for screening numerous clones of hybridoma cell lines for antibody production.

In the examples, below, the elaboration and validation of the sensitive and specific capture ELISA of the present invention is described for antibodies to HPV 16 and 18 E6 and E7 proteins based on the GST-glutathione interaction. This ELISA-type is highly sensitive and specific and has the potential to be readily adapted for a large variety of protein antigens.

### SUMMARY OF THE INVENTION

A novel method is described for the detection and measurement of antibodies in biological fluids such as serum. This newly developed enzyme-linked immunosorbent assay (ELISA) system uses a second binding partner of a binding pair, preferably glutathione, crosslinked to casein as capture protein to bind recombinant protein antigens fused to an N-terminal first binding partner of the binding pair, preferably glutathione S-transferase (GST). The method not only allows efficient and specific detection of antibodies in biological samples but, in addition, simple and efficient immobilization and one-step purificaton of overexpressed recombinant antigens even from crude lysates on ELISA plates coated with, e.g., glutathione casein. Several antigens can be tested in parallel under the same conditions without the need to biochemically purify or renature the proteins, An additional TAG, e.g. undecapeptide epitope, fused, in a particular embodiment, to the C-terminus of each antigen permits the detection and quantification of any full-length protein antigen bound to the ELISA plate with one single monoclonal antibody. In the examples, the ELISA system was applied with four antigens to detect antibodies against E6 and E7 proteins of human papillomavirus types 16 and 18. Antibody reactivities of sera from patients with cervical carcinoma and healthy individuals were in good agreement with those determined using a previously established capture ELISA with biochemically purified and renatured proteins as antigens although the capture ELISA of the present invention was more sensitive with no loss of specificity. The capture ELISA of the present invention was successfully adapted to provide standardized antibody assays for a series of further viral and nonviral protein antigens including the E1, E2, E4 and L1 proteins of HPV types 16 and 18, the E2, E4 and L1 protein of HPV type 6b, the M, N, P and X proteins of Borna Disease Virus (BDV) and bacterial diphteria toxin. The capture ELISA of the present invention could easily be adapted to many other protein antigens from other viruses, bacteria, specific tumour antigens or any other protein for which antibody diagnostics are relevant.

The capture ELISA of the present invention is not only useful for detecting particular antibodies in samples like serum but can also detect and quantify antibodies in biological fluids other than serum such as plasma, urine, saliva, lymph, tears, milk, semen, spinal fluids, ascites, peritoneal and other effusions and vaginal secretions and also to laboratory fluids such as tissue culture supernatans or cell lysates. Antibodies of any immunoglubulin class or subclass and of any animal species can be detected when the appropriate species and class/subclass-specific immunoglobulin detection reagent is used.

Another aspect of the present invention is to provide a test kit for the rapid screening and detection of antibodies in biological fluids and tissues such as serum which is based on the capture ELISA of the present invention.

### BRIEF DESCRIPTION OF THE FIGURES

FIG. 1: Schematic illustration of the GST capture ELISA

Plates are coated with glutathione-casein which mediates indirect binding to the plate of an antigen via N-terminally fused glutathione S-transferase (GST). An additional undecapeptide (TAG) derived from the C-terminus of the SV40 large T-antigen is C-terminally fused to permit the detection and quantification of any bound full-length antigen by the same monoclonal TAG-specific antibody. Human Immunoglobulin G (IgG) bound to the antigen is detected with a secondary human IgG-specific antibody coupled to horseradish peroxidase (HRP).

FIG. 2: Expression and solubility of GST-X-TAG fusion proteins

E6 and F7 proteins of HPV types 16 and 18 were expressed as GST-X-TAG fusion proteins in E. coli BL21 cells. Proteins from equal amounts (20 μg total lysate protein/lane) of total (T) or cleared (S) (supernatants of 30000×g for 30 min) lysates were separated by gel electrophoresis and stained with colloidal Coomassie G-250 M, molecular weight marker with molecular mass in kDa indicated on the left; wt, total lysate of wild-type BL21. Arrowheads indicate the slightly differing positions of the various GST-X-TAG fusion proteins. Note the gel-drying artefact in lane 5 between 55 and 66 kDa.

FIG. 3: Dose-response curve for coating ELISA plates with glutathione-casein

ELISA plates were incubated overnight (4° C., 100 μl/well) with the indicated amounts of glutathione-casein in 50 mM carbonate buffer, pH 9.6. Binding of GST-TAG as detected by anti-TAG monoclonal antibody was used to quantify bound glutathione-casein. Absorbance values are expressed in milliunits (mu).

FIG. 4: Dose-response curves for GST-X-TAG binding to glutathione-casein coated ELISA plates

Serial dilutions blocking buffer of cleared lysates from bacteria overexpressing GST-X-TAG were incubated for 1 h at 4° C. in wells of ELISA plates coated with 200 ng/well of glutathione-casein. Bound GST-X-TAG fusion protein was detected via the C-terminal TAG epitope as shown in FIG. 3.

FIG. 5: Efficiency of one-step purification of GST-X-TAG on glutathione-casein coated plastic surfaces

Plates coated with 2 ng/μl glutathione-casein (lanes 1-8), were blocked with 2 μg/μl unmodified casein (lanes 2-8) or left without blocking (lane 1) and were incubated with cleared bacterial lysates diluted to 250 ng/μl total lysate protein in blocking buffer. The lysates were from wild-type bacteria (lane 3), from bacteria overexpressing GST-TAG (lane 4), GST16E6TAG (lane 5), GST16E7TAG (lane 6), GST18E6TAG (lane 7) or GST18E7TAG (lane 8). Bound proteins were eluted from washed plates with denaturing SDS sample buffer, separated by gel electrophoresis and stained with silver. Lanes contain eluted protein from one well of a microtitre plate. In lane 9, total cleared lysate (100 ng total lysate protein) from bacteria overexpressing GST18E7TAG was electrophoresed. M, molecular weight marker with molecular mass in kDa indicated on the left.

FIG. 6: Reactivity of sera with HPV proteins determined by GST capture ELISA and TAG capture ELISA

Sera from 79 patients with invasive cervical cancer were analyzed separately for antibodies to E6 and E7 proteins of HPV types 16 and 18 in two ELISA formats. Each graph shows the specific absorbance values for one antigen. For each serum, the result from GST capture ELISA is plotted on the ordinate and that from TAG capture ELISA on the abscissa. The linear regression line and the R² value as measure of assay agreement are given. Antibody-positive and -negative sera were grouped according to a cuttoff value (dashed lines) calculated for each antigen and assay format from the control group. The degree of concordance of positive and negative test results of the cervical cancer sera was calculated as a kappa value with 95% confidence interval (95% CI) from 2×2 tables (inset).

### DETAILED DESCRIPTION OF THE INVENTION

The present invention relates to a method for the detection and/or quantification of a first antibody in a sample, comprising the steps of:

(a) coating a binding surface or support with a first binding partner of a binding pair crosslinked to casein to form a coated surface;

(b) incubating the coated surface of step (a) with an antigen fused to the second binding partner of said binding pair;

(c) incubating the complex obtained in step (b) with the sample containing said first antibody;

(d) incubating the complex obtained in step (c) with a second labelled antibody; and

(e) detecting the first antibody bound to the complex of step (c) or determining the amount of first antibody bound to the complex of step (c) by directly or indirectly determining the label of the second labelled antibody.

The person skilled in the art knows suitable binding pairs useful in the method of the present invention. In principle, the aim of the present invention could be reached by use of binding pairs, in which one component is a binding protein used as fusion partner (as GST is used in the Examples, below) with the proteinous antigen and the other component is a small molecule binding with high affinity to the binding protein (as glutathione is used in the Examples, below) and that can be chemically cross-linked to a carrier protein like casein. Examples of suitable binding pairs are glutathione/GST, maltose/maltose-binding protein, biotin/streptavidin etc. For the capture ELISA of the present invention the binding pair glutathione/GST is preferred.

The term “antibody”, as used herein, includes naturally generated antibodies as well as various forms of modified or altered (engineered) antibodies, such as an intact immunoglobin, an Fv-fragment containing only the light and heavy chain variable regions (V_{L} and V_{H}), an Fv-fragment linked by a disulfide bond (Brinkmann et al., PNAS USA 90 (1993), 547-551), an Fab-fragment containing the variable reagions and parts of the constant regions (Fab)′2, dimeric Fabs or trimeric Fabs, which can be multivalent and/or multispecific, a single-chain antibody (ScFv) (Bird et al., Science 242 (1988), 424-426), single-chain multimers (diabodies, triabodies, tetrabodies etc.) which can be multivalent and/or multispecific. The antibody may be of animal (e.g. mouse or rat) or human origin or may be chimeric (Morrison et al., PNAS USA 81 (1986), 6851-6855). The term “chimeric antibody” refers to a hybrid immunoglobulin in which the original murine variable regions are preserved and the constant regions are switched for those of a human antibody. The antibody also may be humanized (Jones et al., Nature 321 (1986), 522-525). The term “humanized antibody” refers to a hybrid immunoglobulin in which the murine residues that conform to specific complementarity determing regions (CDRs) and others of possible structural relevance are transplanted to a human antibody framework. As used herein, the term “antibody” also includes whole antibodies or fragments, which have been fused to radioisotopes, drugs, toxins, enzymes, biosensor surfaces etc. or which have been modified by the addition of a PEG molecule.

The person skilled in the art can crosslink the first binding partner, e,g. glutathione, to a protein like casein according to well known methods, e.g. the method described in the Examples, below, and couple (covalently or noncovalently) this complex to the binding surface or support according to well known methods. The coated surface is exposed to an antigen fused to, e.g. GST, as the second binding partner. Such fused antigen is preferably obtained as a second binding partner/antigen-fusion protein by recombinant expression using a suitable host cell, such as a bacterium, yeast, mammalian or insect cell. A preferred procaryotic host cell is E. coli, a preferred eucaryotic expression system are insect cells or established mammalian cell lines providing the appropriate posttranslational modifications for the specific antigen. For carrying out the method of the present invention it is not required that the antigen fused to the second binding partner is in pure form. It can be applied as a crude or cleared lysate of the host cells used for expression of the antigen. Preferably, the antigen fused to the second binding partner will be present on the solid phase in high, saturating concentrations so that a maximal quantity of antibody present in the sample may be bound. After separating the complex of the first binding partner bound to the surface or support and second binding partner/antigen from unbound material, the solid phase can be exposed to the sample containing the first antibody capable of specifically binding to the captured antigen and, then, with a second labelled antibody which is capable of specifically binding to the first antibody. In this way, label is bound to the solid phase only if the first antibody was present in the sample, e.g. a serum sample. Finally, the first antibody is detected by directly or indirectly determining the label of the second antibody. When appropriate, (a) washing step(s) using suitable buffers and suitable blocking agents suppressing unspecific binding of first and/or second antibody can be included in the method of the present invention described above.

Well known binding surfaces or supports are useful for the method of the present invention, e,g. microtiter plates, small agarose beads, magnetic beads, nitrocellulose paper, nylon filters, suitably treated glass or plastic surfaces etc.

Preferably, the second labelled antibody is a purified polyclonal antibody or a monoclonal antibody, e.g., specific for immunoglobulin (Ig) or a Ig class (IgG, IgM, IgA, IgE, IgD) or Ig subclass (such as human IgG1, IgG2, IgG3, IgG4) from humans or from any Ig-producing animal species. A wide variety of labels can be used for the second antibody, such as radioisotopes, enzymes, fluorescers, chemiluminescers, spin labels, and the like. Enzyme labels may be detected by conventional visualization techniques, e.g., production of soluble or precipitating colored dyes, chemiluminescence, fluorescence, or the like using automated or nonautomated detection devices.

There are many important potential uses for the simple detection of antibody titers in biological fluids, e.g., in serum. Normal plasma from normal or diseased donors can be screened for higher than normal titers of naturally occurring antibodies to known pathogens to identify specific present or past infections. One could easily test sera from humans or animals that have been vaccinated with a particular vaccine, e.g., rabies virus diphteria toxin, etc. and quickly determine if titers were sufficiently elevated to give protection against infection or disease. On the other hand titers of antibodies to viruses known to be associated with the development of particular cancers, e.g. HPV, can be determined in order to show whether there is a predisposition for or presence of the particular type of cancer or precursor lesion. Also, infections with Borna Disease Virus potentially associated with psychiatric disease in Humans could be diagnosed. In tumour tissues tumor-specific antigens are frequently expressed and patients may react with antibody responses to such antigens; diagnosis of such antibodies could be important for the diagnosis or management of tumor disease. The assay of the present invention may lend itself to epidemiological studies in regions were a particular pathogen infects one or more species endemic to that region. A field test kit for detecting serum antibody levels would be amenable for accurately determining particular species involved and rates and spread of infection and could be an important tool in control efforts. Finally, one could also imagine many other in vitro applications for an ELISA of the present invention that could detect antibody levels from experimental tissue culture media or purification samples or the detection of antibody level in biological fluids other than serum such as urine, lymph, spinal fluid etc.

The capture ELISA of the present invention is not limited to pure protein antigens. By use of efficient eukaryotic, expression systems and appropriate, especially mammalian cells, protein antigens carrying the appropriate posttranslational modifications of the natural antigen such as glycosylation, phosphorylation could be produced and used directly from the cell lysate as ELISA antigens. Moreover, the standardized format of the antigen production and presentation in the capture ELISA of the present invention could be adapted to spot large series of antigens in defined geometry on first binding partner/casein coated solid supports (“antigen arrays”) and would allow to determine the antibody status of an individual sample to many antigens in parallel (“antibody profiling”) in a single reaction and manipulation.

In a preferred embodiment of the method of the present invention, said first binding partner is glutathione and the second binding partner is GST.

In a further preferred embodiment of the method of the present invention, the second binding partner, preferably GST, is fused to the N-terminus of the antigen.

In a particularly preferred embodiment, the antigen fused to the second binding partner is additionally fused to a TAG, wherein the TAG is located at the terminus opposite to the terminus fused to said second binding partner. This approach allows to quantify the amount of antigen bound to the support and, in case that the TAG is at the C-terminus of the antigen, to quantify the amount of only full-length antigen bound to the surface coated with the first binding partner. Preferably, for obtaining an antigen additionally comprising at one of its ends a TAG, a DNA is used for recombinant expression containing a sequence encoding the TAG which is located in frame immediately upstream or downstream (depending on the location of the second binding partner) of the sequence encoding the antigen.

Suitable TAGs are known to the person skilled, e.g. the undecapeptide KPPTPPPEPET from the C-terminus of the SV40 large T-antigen, the EQKLISEEDL peptide from the c-myc protein, the synthetic FLAG epitope peptide DYKDDDDK and corresponding anti-TAG-antibodies are commercially available.

In an even more preferred embodiment of the method of the present invention, the binding surface or support is a microtiter plate well.

In the most preferred embodiment of the method of the present invention, the second antibody is labelled with horseradish peroxidase, alkaline phosphatase, biotin, fluorescently or radioactively. One could envision the use of the ELISA of the present invention in kit form. Antigens against specific antibodies (fused to the second binding partner) could be precoated on plates followed by blocking of noncoated solid surface. These plates would then be stable, and could be stored for several months. Peroxidase labelled antibody is also stable for several months at 4° C. Test samples could be added anytime within this window of reagent stability.

Accordingly, the present invention also relates to a test kit for use in an immunoassay to detect a first antibody to an antigen in a sample, comprising:

(a) a support having a surface coated with a first binding partner of a binding pair crosslinked to casein;

(b) an antigen fused to the second partner of said binding pair and bound to said coated surface; and

(c) a purified second labelled antibody that is capable of specifically binding to said first antibody.

As regards particular embodiments of the test kit and the purification method of the present invention reference is made to the above embodiments of the capture ELISA of the present invention

### EXAMPLE 1

### General Methods

(A) Preparation of Glutathione Casein

Casein (Sigma, Deisenhofen, Germany) at a concentration of 5 mg/ml in phosphate-buffered saline (PBS) was incubated for 15 min at RT with 0.4 mM N-ethylmaleimide (NEM) (Sigma) to block the single cysteine residue in casein. Thereafter, 4 mM sulfosuccinimidyl 4-[p-maleimidephenyl]butyrat (SSMBP) (Pierce, Rockford, Ill.) was added as a crosslinker and the reaction proceeded for 30 min a room temperature. Free SSMBP and NEM were separated from casein by size exclusion chromatography on PD10 columns (Pharmacia, Freiburg, Germany). The protein fraction was then supplemented with 10 mm glutathione (Sigma) and the coupling reaction was executed for 1 h at RT. The glutathione-casein was separated from unbound glutathione by gel filtration with PD10, using PBS as buffer and stored at −20° C. in small aliquots.

(B) Recombinant Proteins

HPV types 16 and 18 E6 and E7 coding sequences fused at their 3′-end in frame to a sequence encoding the terminal undecapeptide (amino acid sequence, KPPTPPPEPET) of the SV40 large T-antigen (TAG) were isolated from “BlueScript” plasmids described previously (Meschede et al., J. Clin Microbiol. 36 (1998), 475-480) and inserted into a pGEX vector (Pharmacia) for expression as GST fusion proteins in E. coli. Briefly, coding sequences for E6TAG and E7TAG of HPVI6 and for E7TAG of HPV18, respectively, were mobilized by digestion with BglII and SalI and ligated into BglII/SalI-digested pGEX4T1 plasmid downstream of the GST domain. HPV18 E6TAG sequences were mobilized by SpeI digestion, fill-in reaction with Klenow fragment and SalI digest and were inserted into pGEX4T3 opened by EcoRI digestion, Klenow fill-in and SalI digestion.

An expression plasmid for GST-TAG was constructed by inserting a fragment coding for the TAG epitope, mobilized by BamHI/SalI-digestion from a “BlueScript” plasmid (Meschede et al., 1998), in an appropriately digested pGEX4T3 plasmid.

E. coli BL21 cells transformed with the pGEX plasmids were grown at 25° C. in Luria Bertani medium containing 1 mM ampicillin. At an OD₆₀₀ of 0.5 recombinant protein expression was induced by adding 0.25 mM isopropyl-β-D-thio-galactoside (IPTG) to the medium. The bacteria were harvested 6 h after induction by centrifugation. Pelleted bacteria were resuspended in PBS containing 2 mM DTT, 1% Triton X-100, complete protease inhibitor cocktail (Roche, Mannheim, Germany) and lysed using a high-pressure homogenizer (Avestin, Ottawa, Canada). Lysates were cleared by centrifugation (4° C., 30 min, 30 000×g) and then stored in small aliquots at −20° C.

GST-TAG protein was purified by glutathione affinity chromatography (standard method as described in the “GST Gene Fusion System Manual” by Pharmacia) and a final gel filtration on a Superdex 75 column (Pharmacia),

(C) GST Capture ELISA

Polysorb plastic plates, 96 wells (Nunc, Roskilde, Denmark), were coated overnight at 4° C. with 200 ng/well of glutathione-casein in 50 mM carbonate buffer, pH 9.6. Thereafter, wells were incubated for 1 h at 37° C. with 180 μl of blocking buffer(0.2%(w/v) casein in PBS, 0.05% (v/v) Tween 20), followed by incubation for 1 h at 4° C. with the cleared lysates from E. coli overexpressing GST-X-TAG proteins diluted in blocking buffer to 0.25 μg/μl total lysate protein. Human sera were diluted 1/50 in blocking buffer containing 0.25 μg/μl total lysate protein from the parental E. coli strain BL21 and incubated for 1 h at 4° C. to block reactivities of the sera with contaminating E. coli proteins. Coated ELISA plates were incubated for 1 h with 100 μl/well of diluted and preincubated serum. Bound human antibodies were detected by donkey anti-human immunoglobulin G (IgG) polyclonal antibody conjugated to HRP (1/10000 dilution in blocking buffer, incubation for 1 h at RT; Dianova, Hamburg, Germany) using tetramethylbenzidene (Sigma) (10 μg/ml in 0.1 M NaAcetate, pH 6.0) as substrate with 0.003% H₂O₂. After 8 min, the enzyme reaction was stopped by adding 50 μl of 1 M sulfuric acid/well and the absorbance at 450 nm was measured. Unless indicated otherwise, all incubations were carried out with 100 μl/well at room temperature. All washing steps to remove unbound reagents were done manually with PBS containing 0.05% (v/v) Tween 20 (complete filling of the wells, five repeats). To detect bound GST-X-TAG antigens via their C-terminal TAG, affinity-purified monoclonal mouse IgG1 anti-TAG antibody KT3 (MacArthur and Walter, Virol. 52 (1984), 483-491) at a dilution of 1/1000 (500 ng/ml) was used, followed by goat anti-mouse Ig HRP (1/10000, Dianova). The absorbance in wells with GST-TAG, as antigen defined the background reactivity of a serum, which was then subtracted from the absorbance with the GST-X-TAG proteins to calculate the specific reactivity of a serum against the antigen (X). The TAG capture ELISA using a monoclonal anti-TAG capture antibody and biochemically purified and renatured yeast proteins as antigens was performed as described (Meschede et al., 1998).

(D) Human Sera

The sera used were collected from patients with clinically diagnosed invasive cervical cancer at the Tanzania Tumor Center, Ocean Road Hospital in Dar es Salaam from 1988 to 1991. Based on the results of the TAG capture ELISA, a panel of 79 sera was selected from this collection to represent 16 antibody negative and 63 sera positive for at least one of the four tested early HPV proteins.

Eighty five sera from healthy individuals, randomly taken out of a larger serum collection (n=1644) considered to be representative of the general adult population of Germany were taken as noncervical cancer controls.

(E) Cutoff Definition and Statistical Methods

The cutoff value to define antibody positive sera was calculated separately for each antigen as the median of the specific absorbance values of all control sera plus three standard deviations excluding positive outliers, as described elsewhere (Muller et al., Virology 187 (1992), 508-514). Briefly, control sera with absorbance values higher than the calculated cutoff value were omitted and the calculation was repeated with the remaining sera. This procedure was repeated until the absorbance values of all remaining sera were below the last calculated cutoff value, which was than used to judge sera as antibody-positive or -negative. All sera were measured at least twice and the median of the absorbance values was taken as the final read out.

To compare both ELISA formats, pairs of absorbance values of the sera were entered into a xy-plot and a linear regression curve using the “least squares” method was calculated with standard software. The quality of the fit is indicated by the value of the coefficient of determination, R². The conformity of the two ELISA in classification of sera as antibody-positive or -negative was judged with Cohen's kappa test (Cohen, Educ. Psychol. Mess. 20 (1960), 37-46), considering values>0.7 as good conformity.

Assay reproducibility for each antigen was tested by entering absorbance values of the same sera measured on two different days into an xy-plot. For all CaCx sera (n=79), R² values were between 0.76 and 0.93; and for antibody positive sera only (n=19-42) between 0.64 and 0.91.

### EXAMPLE 2

### Conjugation of Glutathione to Casein

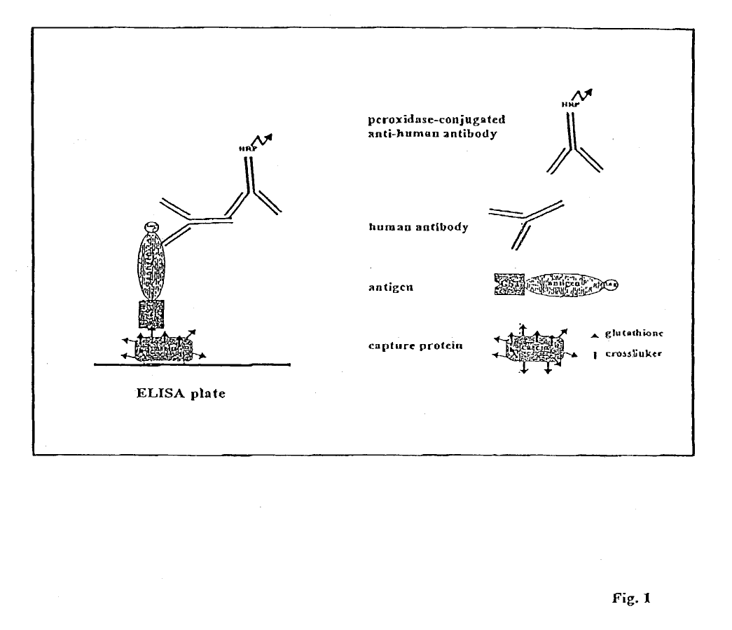

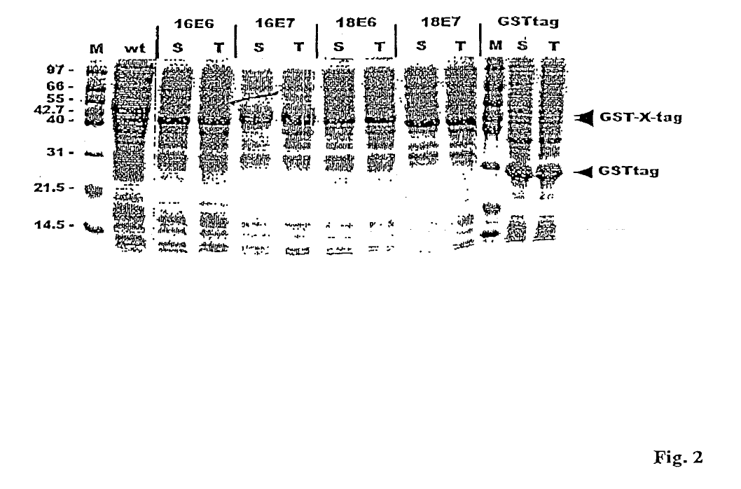

In the GST capture ELISA of this Example, glutathione casein coated to the solid support captures the antigen through the N-terminal GST-part of the fusion protein (see FIG. 1).

The milk protein casein was chosen as carrier protein for the glutathione residues since in previous investigations it was successfully used as a low background ELISA blocking reagent. It mainly consists of α- and β-casein, both of which have a molecular mass of about 25 kDa, one single cysteine and 12 or 15 lysine residues, respectively. Since it is very important to conserve the γ-glutamly-group of glutathione for the interaction with GST crosslinking of glutathione should occur through the sulphydryl-group of its cysteine. To achieve binding of the heterobifunctional crosslinker SSMBP to casein through its aminoreactive succinimidyl group, the single cysteine in casein was blocked with N-ethyl-maleimide (NEM). The final reaction with glutathione could then proceed specifically between the sulphydryl-reactive maleimide group of the crosslinker and the cysteine of glutathione. Thus, casein with a maximum number of functional glutathione residues was generated.

### EXAMPLE 3

### Expression of ELISA Antigens as GST-X-TAG Fusion Proteins

Expression of the E6 and E7 proteins of HPV types 16 and 18 as GST-X-TAG fusion proteins in E. coli yielded high amounts of full-length recombinant protein (FIG. 2). Of the total E. coli lysate proteins, the recombinant GST fusion protein accounted for 16% for the E6 proteins, 25% for the E7 proteins and 30% for GST-TAG as determined by densitometric quantification of the coomassie-stained protein bands with “Image Quant” (Molecular Dynamics, Sunnyvale, Calif., USA). For all five recombinant proteins, the vast majority of the fusion protein expressed was soluble and remained in the supernatant after centrifugation at 30 000×g for 30 min.

### EXAMPLE 4

### Optimization of GST Capture ELISA Conditions

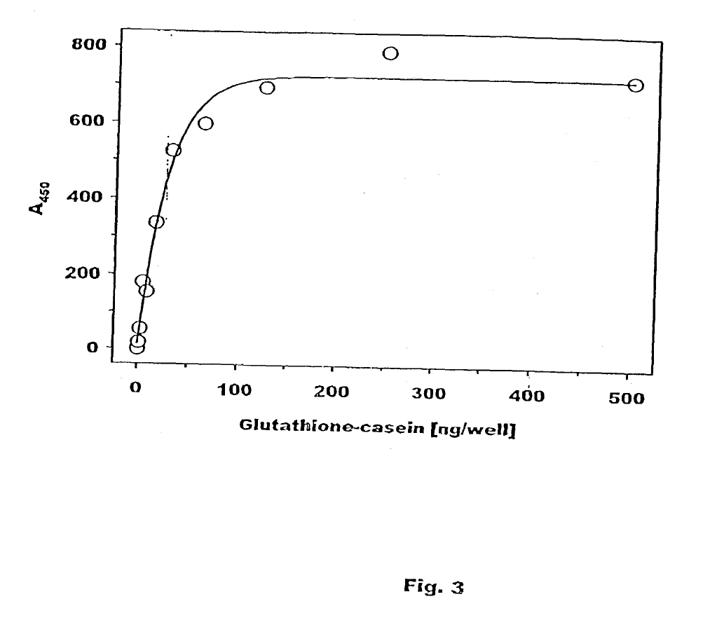

To determine the amount of glutathione casein needed to coat the plastic surface under saturating conditions ELISA plates were coated with 0 to 500 ng/well glutathione casein. Coating was then quantified by an ELISA reaction using purified GST-TAG as antigen and mouse anti-TAG antibody, followed by goat anti-mouse Ig HRP as the detection system. With 125 ng of glutathione casein/well, a plateau of the anti-TAG signal was reached (FIG. 3). In all further experiments, 200 ng of glutathione casein/well were used for coating.

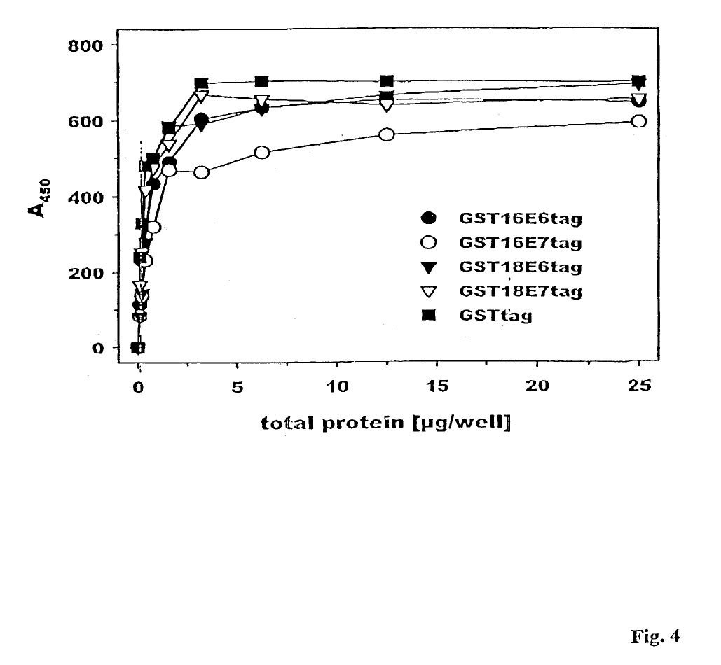

Next, it was determined how much cleared lysate from E. coli overexpressing the GST-X-TAG proteins was needed to saturate the binding capacity of the glutathione casein-coated plates (FIG. 4). Bound GST-X-TAG protein was again quantified via its C-terminal TAG epitope, which selectively detects full-length GST-X-TAG proteins. With all five GST-X-TAG containing lysates, maximal binding of the respective antigen was reached at 25 μg total lysate protein/well. The plateau absorbance levels of all five proteins were similar, indicating that similar amounts of each full-length GST-X-TAG protein were bound irrespective of the inserted antigen X.

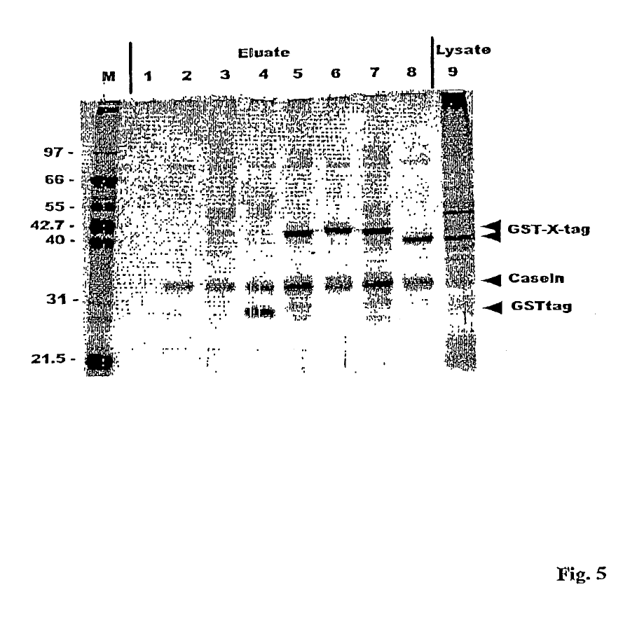

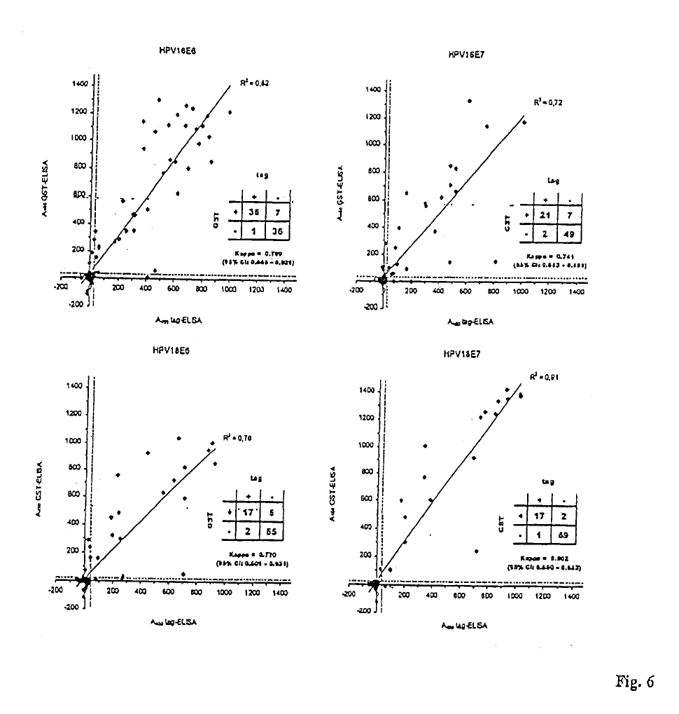

To determine the efficiency of one-step purification of GST-X-TAG proteins from lysates on glutathione casein-coated plates, all bound proteins were elated with a denaturing and reducing buffer, separated by electrophoresis and stained with silver (FIG. 5). A strong enrichment of highly purified GST-X-TAG proteins was found with only minor bacterial protein contaminations. The electrophoretic mobility of casein was lower than expected from the known Mᵣ. To block possible antibody reactions with residual bacterial proteins in the ELISA, the sera were incubated with lysate from wt E. coli BL21.

### EXAMPLE 5

### Validation of GST Capture ELISA to Detect Antibodies to HPV 16 and 18 E6 and E7 Proteins in Comparison with a Previously Established TAG Capture ELISA

Sera from 79 patients with clinically diagnosed invasive cervical cancer with a high E6/E7 antibody prevalence and from 85 healthy control subjects presumably seronegative were analyzed by the GST capture ELISA described above for antibodies against E6 and E7 of HPV types 16 and 18. Results were compared with those obtained with a previously established TAG capture ELISA, which is based on biochemically purified and renatured HPV TAG fusion proteins from yeast and anti-TAG antibody as capture system (Meschede et al., 1998). For all four antigens, comparison of the absorbance value pairs obtained for each serum from the cervical carcinoma group by the two ELISA formats showed agreement of the results with R² values of linear regression ranging from 0.72 to 0.91 for the different antigens (FIG. 6). To group sera as antibody-positive or -negative for each antigen and assay format, cutoff values from the absorbance values obtained with TAG healthy control sera were calculated. The cutoff absorbance values for 16E6, 16E7, 18E6 and 18E7 were 31, 30, 22, 24 milliunits (mU) in the GST capture ELISA and 32, 27,41, 31 mU in the TAG capture ELISA. Agreement of the positive/negative classifications of the cervical cancer sera alone by the two ELISA formats was good with kappa values ranging between 0.741 and 0.802 (insets in FIG. 6). Kappa values increased for 16E6, 16E7, 18E6 and 18E7 to 0.866 (95% CI: 0.775-0.956), 0.753 (95% CI: 0.615-0.891), 0.805 (95% CI: 0.666-0.945) and 0.909 (95% CI: 0.806-1.011) when the control sera were included in the calculation. Overall, more sera positive in the GST capture ELISA were found than in the TAG capture ELISA. With each of the four antigens, 2-7 sera (mean 5.3) from the CaCx-croup (n=79) reacted positive in the GST capture ELISA, but negative in the TAG capture ELISA whereas only 1 or 2 sera (mean 1.5) reacted positive in one of the four TAG capture ELISA, but negative in the GST capture ELISA. All of these sera exhibited absorbance values below 415 mU, with most of them being of low reactivity with less than 110 mU. No differences in positive/negative classification were observed with strongly reactive sera (>415 mU) in any ELISA format. All 85 control sera were negative with all four antigens in the TAG capture ELISA, whereas in the GST capture ELISA, 2 sera were positive with 16E7, one with borderline reactivity (43 mU, cutoff 30 mU) and the other serum with moderately low reactivity (107 mU). Overall, with the GST capture ELISA in the control group two, (weak) positive reactivities in 4×85=340 reactions (0.6%) compared to 0% with the TAG capture ELISA were found, whereas in the CaCx-group 21 positive reactions in 4×79=316 reactions (6.6%) were detected additionally and six (1.9%) positive with the TAG capture ELISA were missed. This indicates that for the GST capture ELISA, in comparison with the TAG capture ELISA there is a substantial increase in sensitivity of antibody detection among patients with HPV-associated cancer and a very small decrease in disease-associated specificity (detection of antibody-positive sera among healthy individuals).

All references cited herein are incorporated by reference in their entirety as if written herein.

Various other examples will be apparent to the person skilled in the art after reading the present disclosure without departing from the spirit and scope of the invention. It is intended that all such other examples be included within the scope of the appended claims.

What is claimed is:

1. A method for the detection and/or quantification of a first antibody in a sample, comprising the steps of:
(a) coating a binding surface or support with a first binding partner of a binding pair crosslinked to casein to form a coated surface;
(b) incubating the coated surface of step (a) with an antigen fused to the second binding partner of the binding pair;
(c) incubating the complex obtained in step (b) with the sample containing said first antibody;
(d) incubating the complex obtained in step (c) with a second labelled antibody capable of binding to the first antibody; and
(e) detecting the first antibody bound to the complex of step (c) or determining the amount of first antibody bound to the complex of step (c) by directly or indirectly determining the label of the second labelled antibody.

2. The method of claim 1, wherein said first binding partner is glutathione and said second binding partner is GST.

3. The method of claim 1, wherein said antigen is additionally fused to a TAG and wherein said TAG is located at the terminus opposite to the terminus fused to said second binding partner.

4. The method of claim 1, wherein said second binding partner is fused to the N-terminus of the antigen.

5. The method of claim 3, wherein said TAG is the undecapeptide KPPTPPPEPET.

6. The method of claim 1, wherein said binding surface or support is a microtiter plate well.

7. The method of claim 1, wherein said second labelled antibody is a purified polyclonal antibody.

8. The method of claim 1, wherein said second labelled antibody is a monoclonal antibody.

9. The method of claim 1, wherein said second antibody is labelled with an agent selected from the group consisting of:
horseradish peroxidase, alkaline phosphatase, biotin and fluorescent label.

10. A test kit for use in an immunoassay to detect a first antibody in a sample, comprising:
(a) a support having a surface coated with a first binding partner of a binding pair crosslinked to casein;
(b) an antigen fused to the second binding partner of said binding pair and bound to said coated surface via the interaction of the first and second binding partners; and
(c) a purified second labelled antibody that is capable of binding to said first antibody.

11. The test kit of claim 10 further comprising washing reagents, incubation reagents, and label substrate.

12. The test kit of claim 10, wherein said first binding partner is glutathione and said second binding partner is GST.

13. The test kit of claim 10, wherein said antigen is additionally fused to a TAG and wherein the TAG is located at the terminus opposite to the terminus fused to said second binding partner.

14. The test kit of claim 10, wherein said second binding partner is fused to the N-terminus of the antigen.

15. The test kit of claim 13, wherein said TAG is the undecapeptide KPPTPPPEPET.

16. The test kit of claim 10, wherein said binding surface or support is a microtiter plate well.

17. The test kit of claim 10, wherein said second labelled antibody is a purified polyclonal antibody.

18. The test kit of claim 10, wherein said second labelled antibody is a monoclonal antibody.

19. The test kit of claim 10, wherein said second antibody is labelled with an agent selected from the group consisting of:
horseradish peroxidase, alkaline phosphatase, biotin and fluorescent label.
